(19) United States
(12) Patent Application Publication
(10) Pub. No.: US 2019/0254823 A1
(43) Pub. Date: Aug. 22, 2019

(54) IMPLANTABLE DEVICE FOR SHAPING AN INTRACORPOREAL WALL, AND ASSOCIATED TREATMENT KIT

(71) Applicant: Mustapha LADJALI, RUEIL MALMAISON (FR)

(72) Inventor: Mustapha LADJALI, RUEIL MALMAISON (FR)

(21) Appl. No.: 16/333,896

(22) PCT Filed: Sep. 15, 2017
(86) PCT No.: PCT/EP2017/073333

§ 371 Date: Apr. 4, 2019

(30) Foreign Application Priority Data
Sep. 16, 2016 (FR) 1658699

(51) Int. Cl. A61F 2/24; A61B 17/04

(52) U.S. Cl. CPC A61F 2/2445; A61B 17/0487; A61B 2017/00243; A61F 2250/001; A61F 2250/0007

(57) ABSTRACT

An implantable device for shaping an intracorporeal wall includes: an elongate element which has a first end, a second end and a central filiform region between the first end and the second end; and a blocking member. The elongate element is mobile in relation to the blocking member between an unlocked position away from the blocking member and a locked position on the blocking member, in which the elongate element is connected to the blocking member. The blocking member includes a mechanism for selective adjustment of the length of the central region protruding towards the second end from the blocking member.

The present invention relates to an implantable device for shaping an intracorporeal wall, including:
an elongate element having a first end, a second end and a central filiform region between the first end and the second end,
a blocking member, the elongate element being mobile in relation to the blocking member between an unlocked position away from the blocking member and a locked position on the blocking member, in which the elongate element is connected to the blocking member.

Such a device is in particular intended to form a valvular annuloplasty device, intended to be implanted advantageously through the endovascular route in order to shape a heart valve such as the mitral valve, the tricuspid valve or the aortic valve.

It applies to other intracorporeal walls in particular to allow the adjustment of the length of a prosthetic valve chord, the closure of an inter-auricular communication (IAC) or an inter-ventricular communication (IVC) or the closing of a periprosthetic leak, etc.

“Annuloplasty” refers to the repair or reconstruction of the native fibromuscular annulus of the valve in question.

In heart valves, the leaflets of the valve are capable of coming into contact by their free edges at the center of the fibromuscular annulus that surrounds the valve. Depending on the nature of the valve in question, the leaflets thus provide the sealing of the valve against a regurgitation of blood from the ventricle toward the atrium or from the aorta into the ventricle.

However, some affections may lead to deformations or expansions of native valve annuluses, resulting in a sealing defect of the leaflets due to insufficient contact between the edges of the leaflets, and as a result, a regurgitation of blood.

One option for treating this problem, as long as the deformation of the annulus is not too great, is to perform a reconstructive re-contouring of the annulus, also called annuloplasty, rather than a complete replacement of the valve. To that end, it is possible to shape the native valve with a prosthetic annulus in order to bring the edges of the leaflets closer to one another and therefore reestablish the sealing of the valve.

An example prosthetic annulus typically used is the Carpentier-Edwards annulus. This annulus comprises a core surrounded by a fabric sheath. The prosthetic annulus delimits a central orifice with dimensions substantially corresponding to the dimensions of the native orifice before the deformation of the native orifice. The orifice delimited by the prosthetic annulus is therefore suitable for restoring the contact between the edges of the leaflets, and therefore the sealing of the valve, as of implantation of the annulus.

Such a prosthetic annulus is not fully satisfactory. Indeed, it is necessary to provide the proper annulus dimension before implantation. Furthermore, once implanted, the shape of the annulus no longer changes. The immediate reestablishment of the sealing of the valve resulting from the implantation of the prosthetic annulus leads to an abrupt increase in the muscular work to be provided by the heart during the cardiac cycle, in particular to eject the blood. Yet this abrupt increase makes the patient's heart more fragile, the latter's muscles no longer being accustomed to supplying such an effort.

Furthermore, the tissues of the native annulus may later relax, causing unsuitability of the prosthetic annulus for the new shape of the heart valve.

Lastly, the implantation of a complete annulus requires a heavy open-heart surgical procedure.

One aim of the invention is therefore to provide a device for shaping an intracorporeal wall that remains suitable over time for a desired shape of the intracorporeal wall, and that is easy to implant.

To that end, the invention relates to a shaping device of the aforementioned type, characterized in that the blocking member includes a mechanism for selective adjustment of the length of the central region protruding towards the second end from the blocking member.

The device according to the invention can further include one or more of the following features, considered alone or according to any technically possible combinations.
the blocking member comprises a fixed base, delimiting a bearing surface on an intracorporeal surface, the base delimiting a passage orifice for the elongate element, the central region of the elongate element protruding outside the blocking member while passing through the passage orifice in the locked position;
the base includes a deflector protruding from the bearing surface away from the selective adjusting mechanism, the deflector guiding the central region of the elongate element from the passage orifice;
the elongate element is provided with at least one blocking stop, able to cooperate with the blocking member in the locked position;
the blocking stop is mounted at the first end or near the first end, and is maneuverable between an axial position for insertion in the blocking member and a transverse position for connecting to the blocking member;
the selective adjustment mechanism includes a winder able to wind part of the central region of the elongate element around a winding axis;
the blocking member comprises a fixed base, delimiting a bearing surface on an intracorporeal surface, the winder comprises a mobile member, mounted rotating relative to the base around the winding axis;
the mobile member defines at least one through passage through which the elongate element passes in the locked position;
the winder includes an element for fastening a maneuvering arm rotatably to the mobile number;
the selective adjustment mechanism includes a unidirectional rotational blocking system for the winder around the winding axis;
the elongate element is provided, at its second end, with a bearing plate on an intracorporeal surface or a fastening clamp on an intracorporeal surface;
in the locked position of the elongate element, the first end and the second end are mounted in the blocking member, the central region forming a loop including a C-shaped tubular member, the elongate element cooperating mechanically with the tubular member to modify the shape of the tubular member by selective adjustment of the length of the central region of the elongate element using the selective adjustment mechanism of the blocking member.
the base includes a bearing patch on the intracorporeal surface, protruding radially, the patch advantageously being deployed spontaneously between a contracted configuration and a radially deployed configuration.
the rotation axis of the mobile member is transverse to the local axis of the filiform central region at the outlet of the blocking member;
the rotation axis of the mobile member is collinear to the local axis of the filiform central region at the outlet of the blocking member;
the winder comprises a rotary shaft and a fastening device on an intracorporeal wall, the rotary shaft being mounted rotatably relative to the fastening device.

The invention also relates to a treatment kit comprising:
a shaping device as defined above;
a device for actuating the selective adjustment mechanism, maneuverable reversibly between an engaged configuration for actuating the selective adjustment mechanism and a configuration detached from the selective adjusting mechanism.

The kit according to the invention can further include one or more of the following features, considered alone or according to any technically possible combinations.
the actuating device includes a catheter, able to be applied over the blocking member, and a mobile arm, able to grasp a fastening element of the selective adjustment mechanism.
it comprises a device for placement of the shaping device, the placement device including a traction guide for the first end of the elongate element, able to be reversibly fixed on a fastening site of the elongate element.

The invention also relates to a method for shaping an intracorporeal wall of a patient, including the following steps:
providing a treatment kit as defined above;
inserting the elongate element through the intracorporeal wall to be shaped;
bringing the blocking member into contact with the intracorporeal wall;
locking the elongate element on the blocking member;
selectively adjusting the length of the central region protruding towards the second end from the blocking member by actuating the selective adjustment device.

Advantageously, the method according to the invention further comprises one or more of the following features, considered alone or according to any technically possible combinations:
the selective adjustment is done using a device for actuating the selective adjustment mechanism inserted into the body, maneuverable reversibly between an engaged configuration for actuating the selective adjustment mechanism and a configuration detached from the selective adjusting mechanism, the method comprising, after the adjustment, the passage of the actuating device into the detached configuration and removal of the actuating device from the patient's body;
the selective adjustment is done using a device for actuating the selective adjustment mechanism inserted by the endoluminal route into the body, maneuverable reversibly between an engaged configuration for actuating the selective adjustment mechanism and a configuration detached from the selective adjusting mechanism, the method comprising, after the adjustment, the passage of the actuating device into the detached configuration and removal of the actuating device from the patient's body;
the method includes, after the removal step, at least one new insertion into the patient's body of a device for actuating the selective adjustment mechanism comprising the passage of the actuating device into the engaged configuration, and at least one new selective adjustment of the length of the central region protruding towards the second end from the blocking member by actuating the selective adjustment device,
the method includes, after the removal step, at least one new insertion into the patient's body by the endoluminal route of a device for actuating the selective adjustment mechanism comprising the passage of the actuating device into the engaged configuration, and at least one new selective adjustment of the length of the central region protruding towards the second end from the blocking member by actuating the selective adjustment device.

The invention also relates to a device for shaping a valvular annulus, including:
a C-shaped tubular member, the ends of the tubular member being separated;
an elongate element having a first end, a second end and a central filiform region between the first end and the second end,
a blocking member, the elongate element being mobile in relation to the blocking member between an unlocked position away from the blocking member and a locked position on the blocking member, in which the elongate element is connected to the blocking member,

characterized in that the blocking member includes a mechanism for selective adjustment of the length of the central region protruding toward the second end from the blocking member, the elongate element cooperating mechanically with the tubular member to modify the shape of the tubular member by selectively adjusting the length of the central region of the elongate element using the mechanism for selective adjustment of the blocking member.

The device may comprise one or more of the features above, considered alone or according to any technically possible combination(s), or one of the features below:
the C-shaped tubular member delimits the central aperture, the central region being engaged at least partially through the central aperture;
the second end of the elongate element is fastened to one end of the C-shaped tubular member, the central region of the elongate element passing through another end of the C-shaped tubular member.

The invention will be better understood upon reading the following description, provided solely as an example and done in reference to the appended drawings, in which:

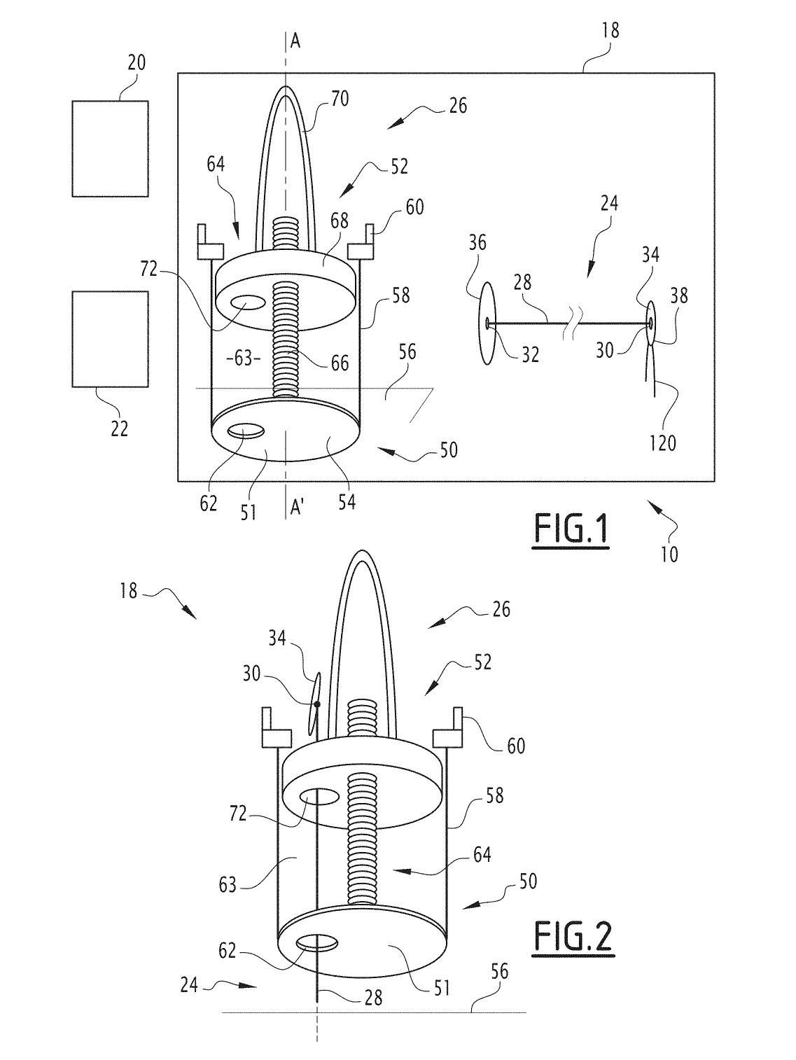

FIG. 1 is a schematic view of a first treatment kit comprising a device for shaping an intracorporeal wall according to the invention;

FIG. 2 is a view of the blocking member of the shaping device, the first end of the elongate element being locked in the blocking member;

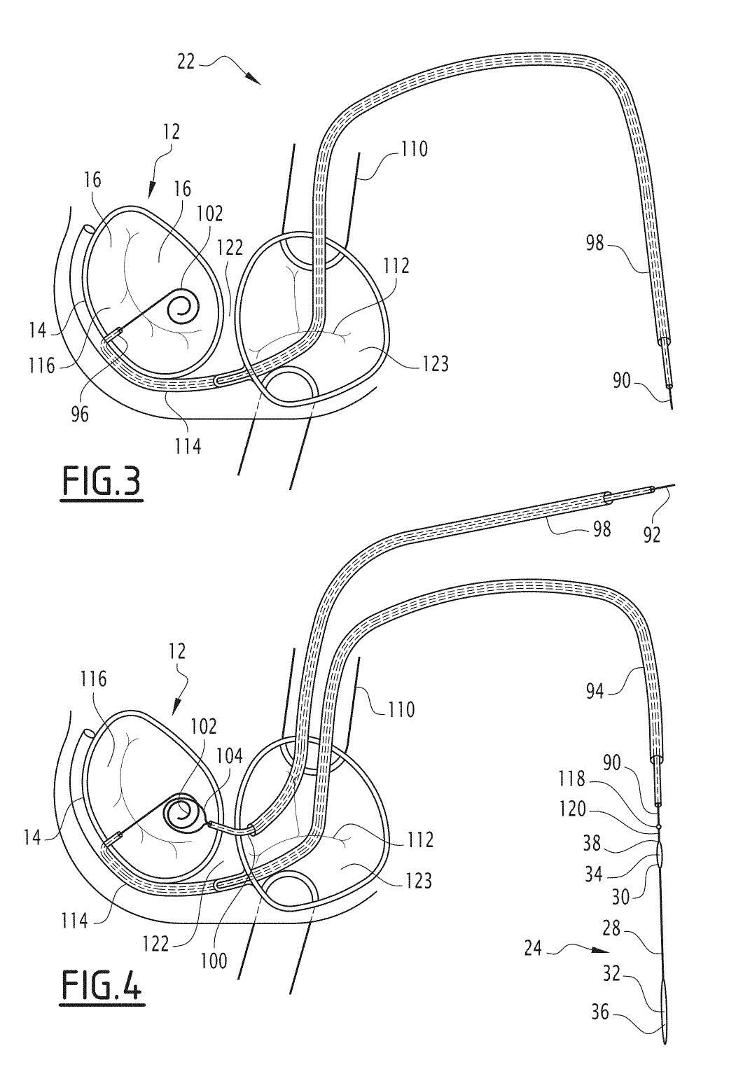

FIGS. 3 to 7 illustrate successive steps of a method for shaping an intracorporeal wall, here the wall of the left atrium around a mitral annulus, using the kit of FIG. 1;

FIGS. 8 to 17 illustrate variants of a shaping device according to the invention;

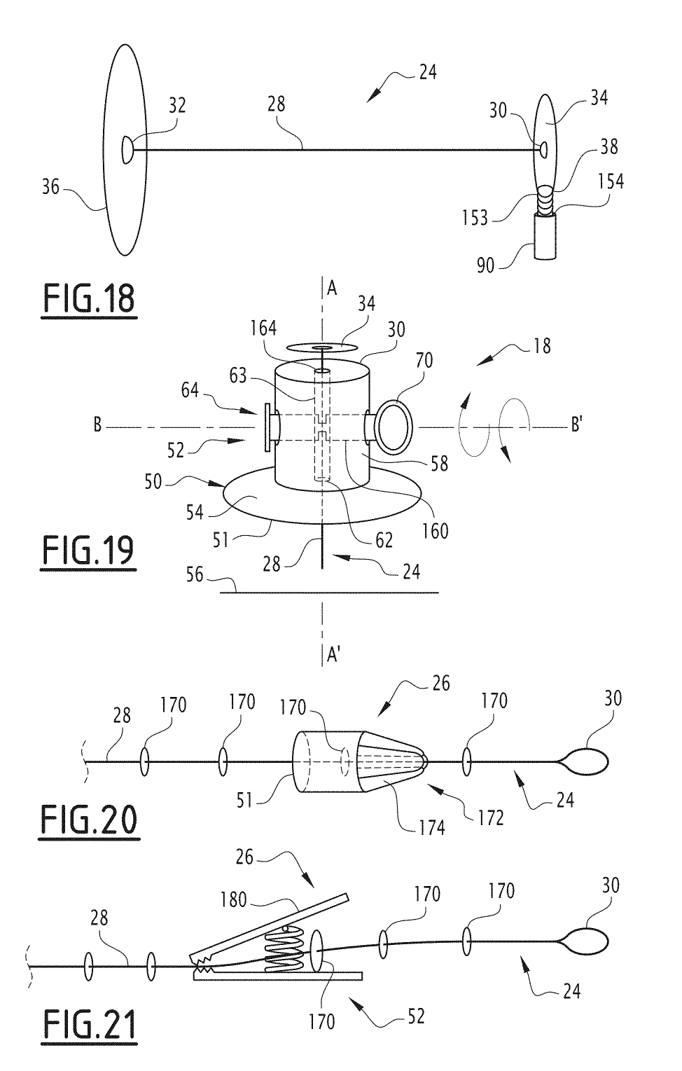

FIG. 18 illustrates a variant of an elongate element for a shaping device according to the invention;

FIG. 19 illustrates a variant of a blocking member for a shaping device according to the invention;

FIGS. 20 to 21 illustrate other variants of shaping devices according to the invention;

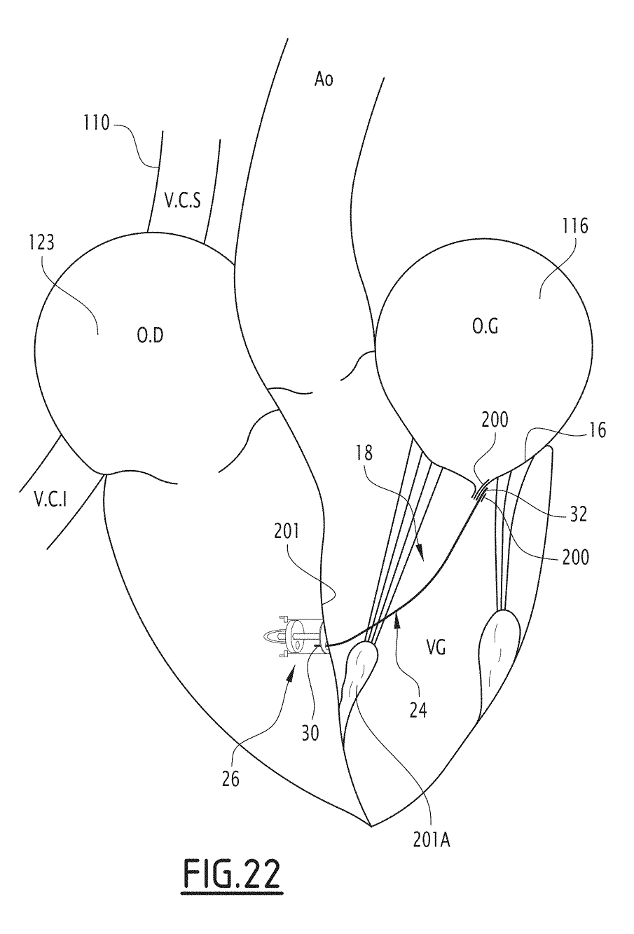

FIG. 22 illustrates a variant of a shaping device according to the invention intended to form a valvular prosthetic chord;

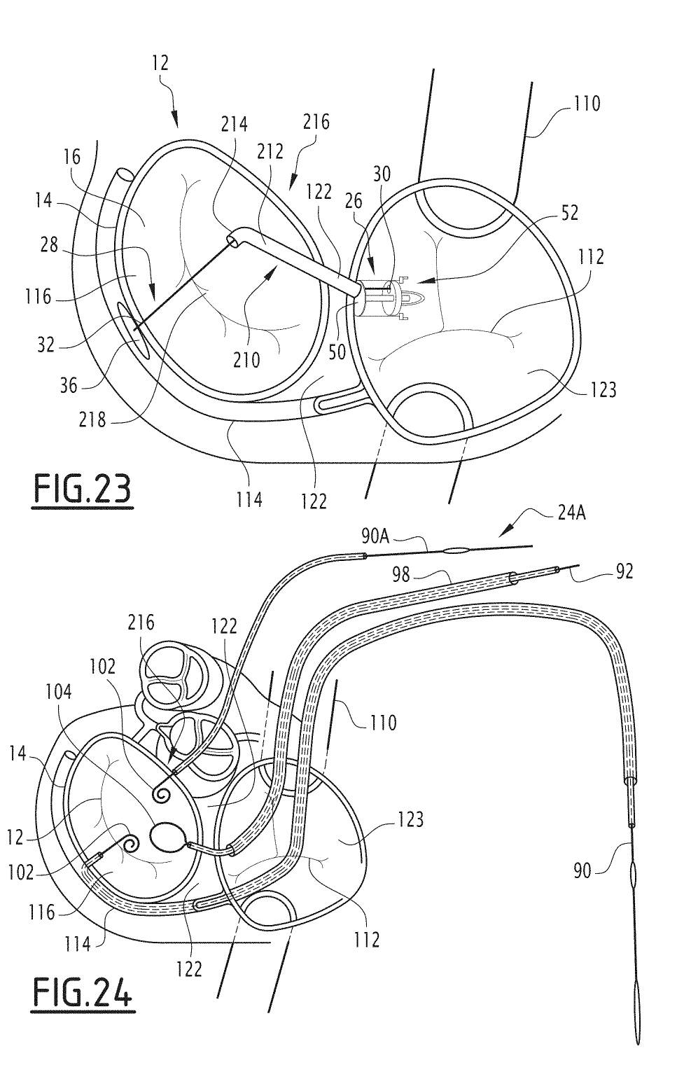

FIGS. 23 to 28 illustrate other variants of shaping devices according to the invention;

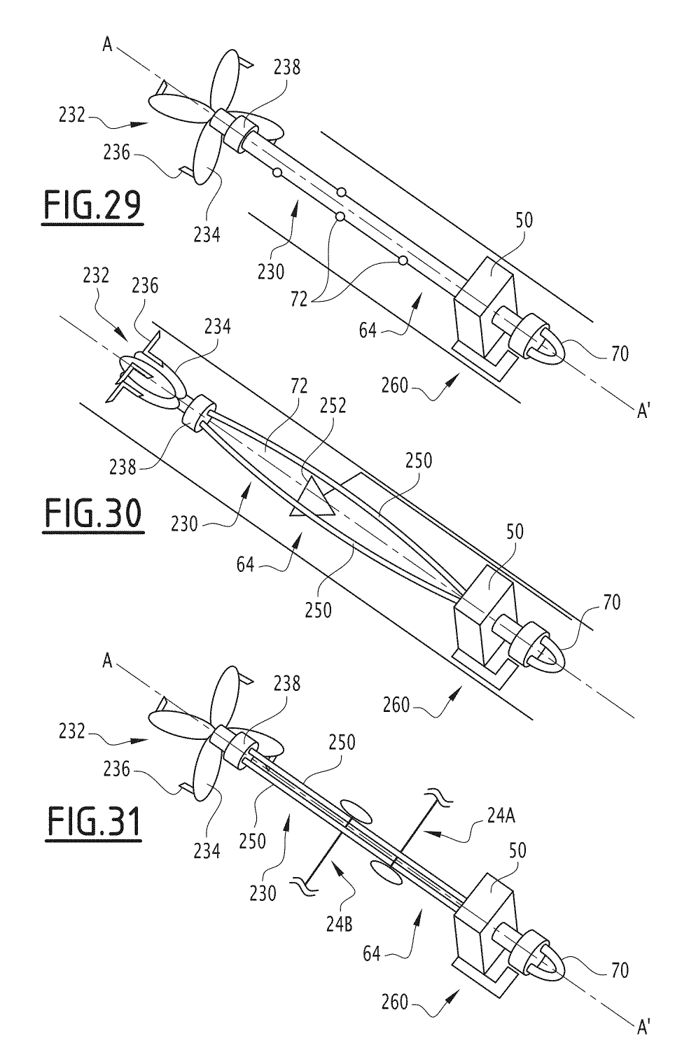

FIGS. 29 to 33 illustrate blocking members of other variants of shaping devices according to the invention.

A first treatment kit 10 according to the invention is illustrated schematically in FIG. 1.

This kit 10 is in particular intended to treated patients suffering from heart disease by valvular annuloplasty. The kit 10 is in particular intended to treat heart valves, particularly the mitral valve 12, visible in FIG. 3, by shaping an intracorporeal wall 14.

The intracorporeal wall 14 here is a wall located around a native annulus from which leaflets 16 of the valve 12 extend. In the case of the mitral valve, the wall 14 is the wall of the left atrium situated around the native annulus. Alternatively, the intracorporeal wall is the native annulus directly.

In reference to FIG. 1, the treatment kit 10 includes an implantable device 18 for shaping the intracorporeal wall 14, a device 20 for actuating the shaping device 18, and a device 22 for placing the shaping device 18 in the patient.

The shaping device 18 comprises a deformable elongate element 24, and a member 26 for blocking the elongate element 24 against an intracorporeal surface. The blocking member 26 is initially disassembled from the elongate element 24, the elongate element 24 being able to be locked in the blocking member 26 during the implantation of the shaping device 18, as will be seen later.

As illustrated by FIG. 1, the elongate element 24 includes a filiform central region 28 defining a first end 30 intended to engage in the blocking member 26 and a second end 32 intended to bear on an intracorporeal surface.

In the example illustrated in FIG. 1, the ends 30, 32 are separated and are mobile separately relative to one another.

The elongate element 24 includes, at its first end 30, a mobile stop 34 for engaging in the blocking member 26. It includes, at its second end 32, a plate 36 for bearing on the intracorporeal surface.

The mobile stop 34 here is in the shape of a grain of rice. It is able to pivot around the first end 30 around at least two axes, between an axial position, oriented axially relative to the local axis of the central region 28 at the first end 30, and a transverse position, oriented transversely relative to the local axis of the region 28 at the first end 30.

The mobile stop 34 is provided with a site 38 for fastening of a traction guide of the placement device 22. The fastening site 38 is off-centered relative to the local axis of the central region 28 at the first end 30.

The bearing plate 36 is able to pivot around the second end 32 around at least two axes. Here, it has a transverse expanse greater than that of the mobile stop 34.

The filiform region 28 connects the mobile stop 34 to the bearing plate 36. It is made up of a wire, able to deform without variation in its total length.

The total length of the filiform region 28 is for example between 0.5 cm and 10 cm.

The blocking member 26 includes a base 50 having a bearing surface 51 on an intracorporeal surface, the base 50 having local axis A-A′, and a mechanism 52 for selective adjustment of the length of the central region 28 protruding from the blocking member 26 toward the second end 32.

In this example, the base 50 includes a stationary plate 54, and a patch 56 protruding radially relative to the stationary plate 54 in order to increase the cooperation area between the base 50 and the intracorporeal surface.

The base 50 further includes a guide tube 58 and a collar 60 for application of the actuating device 20.

The stationary plate 54 in this example defines an orifice 62 for passage of the elongate element 24.

The guide tube 58 protrudes away from the patch 56 from the stationary disc 54 along the axis A-A′. It defines an inner passage 63 for receiving the adjustment mechanism 52.

The application collar 60 is arranged around the free edge of the guide tube 58.

The selective adjustment mechanism 52 here includes a winder 64 able to wind part of the central region 28 of the elongate element around a winding axis collinear with the axis A-A′.

The winder 64 in this example comprises a threaded rod 66, a rotary member 68, mounted on the threaded rod 66, and a fastening element 70 intended to cooperate with the actuating device 20 in order to rotate the rotary member 68.

The threaded rod 66 protrudes along the axis A-A′ inside the guide tube 58.

The rotary member 68 is mounted moving on the threaded rod 66 rotating about the axis A-A′ and moving in translation along the axis A-A′. It defines a through passage 72 for engagement of the central region 28, intended to rotate the central region 28 and the winder about the rod 66.

The through passage 72 is off-centered relative to the axis A-A′. It emerges axially on either side of the rotary member 68.

As will be seen below, the elongate element 24 is able to be inserted in the blocking member 26, from an unlocked position, initially separated from the blocking member 26, toward a locked position, in which the first end 30 of the blocking member 26 is locked on the blocking member 26.

To that end, the mobile stop 34 is able to be inserted successively in its axial position through the passage orifice 62, then through the through passage 72, and to enter its transverse position to stay bearing against the rotary member 68 in the locked position.

As illustrated by FIG. 2, the filiform central region 28 is then engaged partially in the guide tube 58.

The rotation of the rotary member 68 in a first direction causes the winding of an increasing length of the central region 28 in the blocking member 26, and the decrease of the length of the central region 28 protruding toward the second end 32 outside the blocking member 26.

On the contrary, the rotation of the rotary member 68 in a second direction causes the unwinding of the central region 28 and the increase of the length of the central region 28 protruding toward the second end 32 outside the blocking member 26.

Thus, the length of the central region 28 protruding toward the second end 32 outside the blocking member 26 is able to vary selectively between 0% and 90% of the total length of the filiform central region 28, for example between 0.3 cm and 9.5 cm.

In the example shown in FIG. 1, the fastening element 70 is formed by a hoop mounted on the rotary member 68 protruding outside the guide tube 58.

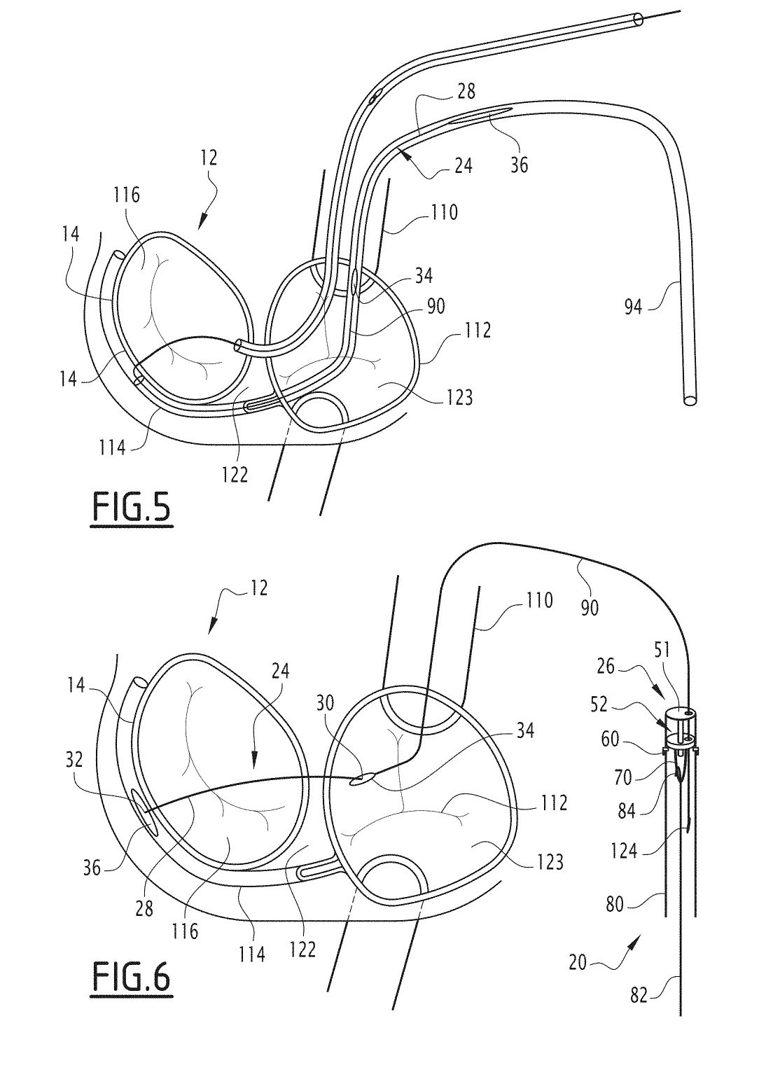

In reference to FIG. 6, the actuating device 20 includes a catheter 80 able to be applied fixedly on the base 50, and a mobile arm 82 received in the catheter 80 in order to grasp the fastening element 70 and rotate it about the axis A-A′.

In the example shown in FIG. 6, the mobile arm 82 is provided at its end with a hook 84 for grasping the hoop.

The actuating device 20 is maneuverable reversibly between an engaged configuration for actuating the selective adjustment mechanism 52 and a configuration detached from the selective adjusting mechanism 52.

In reference to FIGS. 3 to 5, the placement device 22 includes a filiform endovascular guide 90 for traction of the elongate element 24 and raising of the blocking member 26. The placement device 22 further includes a traction member 92 of the intake guide 90.

It also includes a first probe 94 engaged on the guide 90, and a first intracorporeal wall passage needle 96 for bringing the guide 90 to the implantation region of the shaping device 18.

The placement device 22 further includes a second probe 98 engaged around the traction member 92 and a second intracorporeal wall passage needle 100 for bringing the traction member 92 to the implantation region of the shaping device 18.

The guide 90 here is able to be bent in a cross shape at its free end 102.

The traction member 92 includes, at its free end, a grasping member 104, for example a lasso, able to grasp the free end 102 of the guide 90 in order to exert a traction force on said end 102.

The implantation and operation of the treatment kit 10 according to the invention will now be described, in reference to FIGS. 3 to 7. This description is done here in the context of the placement of a shaping device 18 for a native valvular annulus of a mitral valve 12. The method could apply similarly to other intracorporeal walls in particular to allow the adjustment of the length of a prosthetic valve chord, the closure of an inter-auricular communication (IAC) or an inter-ventricular communication (IVC) or the closing of a periprosthetic leak.

Initially, the elongate element 24 and the blocking member 26 are provided disassembled outside the patient's body, the elongate element 24 being in its unlocked position relative to the blocking member 26.

In reference to FIG. 3, the guide 90 is introduced into the patient's body, through the endoluminal route. In the example shown in FIG. 3, the guide 90 is successively passed through the superior vena cava 110, opposite the tricuspid valve 112, through the coronary sinus 114, then through the wall 14 of the left atrium 116 near the native annulus delimiting the mitral valve 12 to enter the left atrium 116.

The free end 102 of the guide 90 is then wound in a cross shape.

In reference to FIG. 4, the elongate element 24 is then fastened to the proximal end 118 of the guide 90. This fastening is for example done by connecting the fastening site 38 on the mobile stop 34 at the proximal end 118 of the guide by a connecting thread 120 (visible in FIG. 1).

Then, the second needle 100 is successively introduced through the superior vena cava 110, and the interatrial septum 122, for the placement of the second probe 98 and the traction member 92.

The traction member 92 is then maneuvered so that the grasping member 104 grasps the free end 102 of the guide 90. The traction member 92 is then manipulated to pull the guide 90 toward the second probe 98, and through it.

The traction on the guide 90 causes the elongate element 24 to rise in the first probe 94, then outside it, as illustrated by FIG. 5. The mobile stop 34 successively passes in the left atrium 116, then through the septum 122 to be inserted in the right atrium 123, opposite the tricuspid valve 112, as shown in FIG. 6.

On the contrary, the bearing plate 36 remains blocked in the coronary sinus 114, bearing against the wall 14 around the native annulus.

This being done, the second probe 98 is removed.

The blocking member 26 is then mounted on the actuating device 20. The free end of the catheter 80 is engaged on the collar 60. The hook 84 is inserted in the hoop of the fastening element 70.

The free end 124 of the guide 90 is successively inserted into the passage orifice 62, then in the through passage 72 in order to allow the blocking member 26 and the actuating device 20 to rise along the guide 90 up to the cavity opposite the septum 122, through the superior vena cava 110.

The mobile stop 34 is placed in its axial position to successively enter the passage orifice 62 and the through passage 72. It then enters its transverse position, which locks the elongate element 24 relative to the blocking member 26. The filiform central region 28 then extends from the blocking member 26 through the septum 122, the left atrium 116, and through the wall 14 to its second end 32.

The length of the central region 28 protruding toward the second end 32 outside the blocking member 26 is then adjusted by maneuvering the arm 82 in order to rotate the rotary member 68 about the axis A-A′. This winds or unwinds part of the central region 28 from the winder 64, in order to adjust the desired length of the central region 28 outside the blocking member 26.

The adjustment of this length adjusts the distance between the blocking member 26 and the bearing plate 36, controlling the deformation of the wall 14 and adjusting it to the desired shape.

Once the adjustment is done, the arm 82 is separated from the fastening element 70. The catheter 80 is detached from the blocking member 26 and is removed from the patient. The guide-90 is then freed from the mobile stop 34 and is removed from the patient.

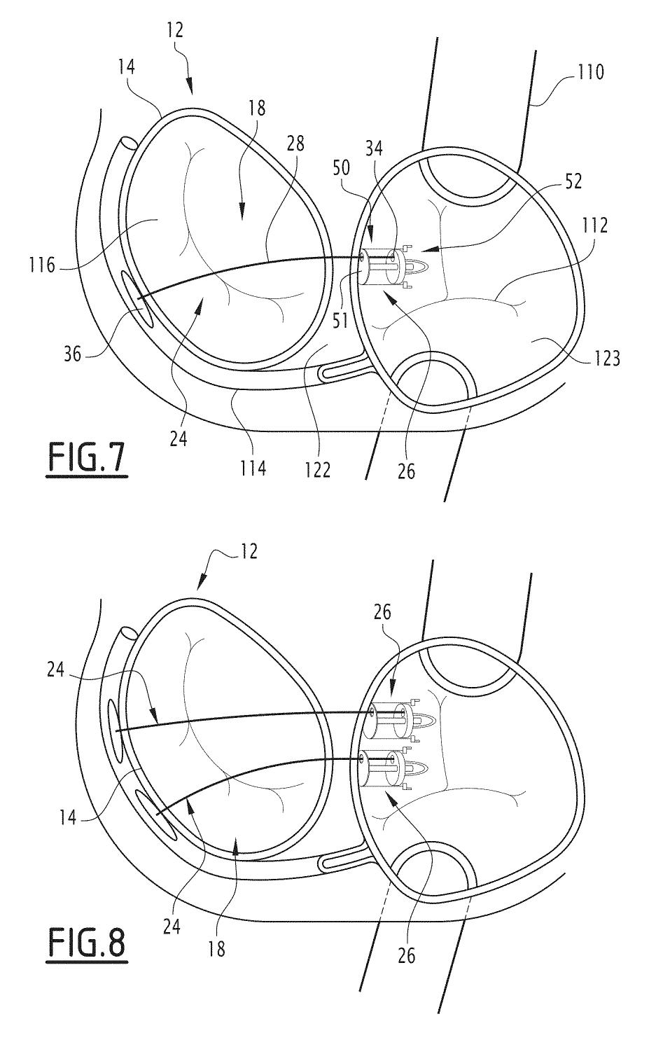

The shaping device 18 is then installed, as illustrated by FIG. 7.

Later, if an adjustment to the shape of the wall 14 is required, the catheter 80 provided with the arm 82 is reintroduced into the patient, through the superior vena cava 110 to reach the blocking member 26. The hook 84 at the end of the arm 82 is then reengaged in the fastening element 70 to maneuver the adjusting mechanism 52, as previously described.

The shaping device 18 according to the invention is therefore particularly easy to place through the endoluminal route and allows an adjustable adjustment of the shaping of an intracorporeal wall 14. This adjustment can be modified easily over time using an actuating device 20 also inserted through the endoluminal route.

This greatly limits the risks for the patient, and guarantees adaptability to the anatomical configuration of the patient, which can change over time.

In the alternative shown in FIG. 8, two shaping devices 18 are placed while bearing, on the one hand, on the wall of the left atrium 116, and on the other hand, on the interatrial septum 122.

This makes it possible to adjust the shaping of the wall 14 precisely.

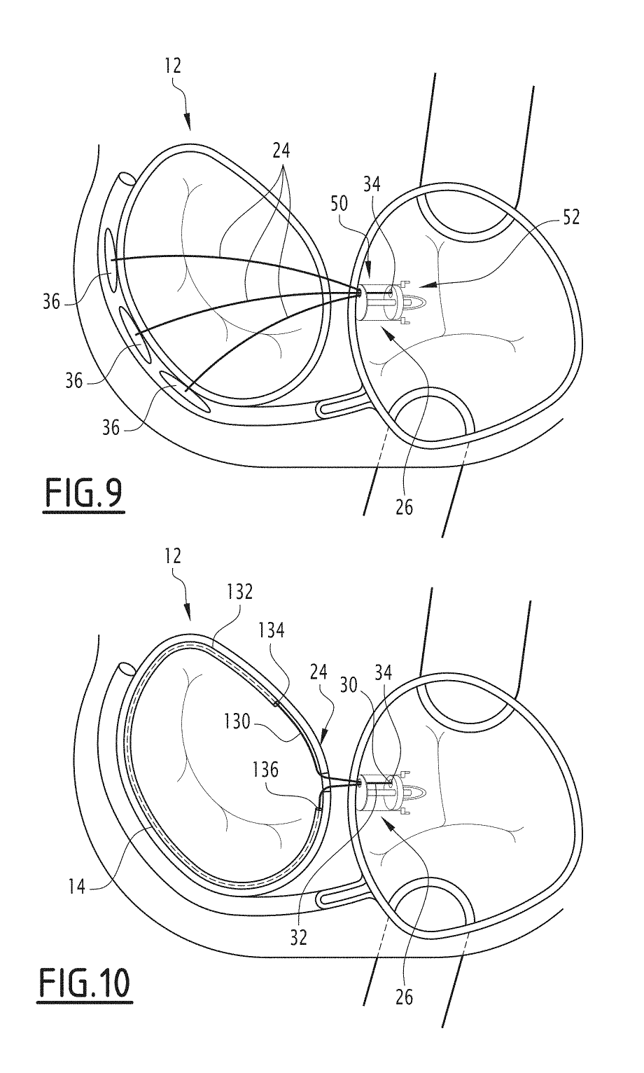

In the variant shown in FIG. 9, the shaping device 18 includes a plurality of elongate elements 24 each having a bearing plate 36. The elongate elements 34 all have a shared first end 30 provided with a single mobile stop 34, inserted into a blocking member 26.

In the variant shown in FIG. 10, the elongate element 24 forms a loop 130. The first end 30 and the second end 32 are each connected to the mobile stop 34, which is inserted into the blocking member 26.

In this example, a tubular member 132 is engaged around the central region 28 in the form of a loop 130.

The tubular member 132 has separated ends 134, 136. Opposite strands of the loop 130 respectively leave the tubular member 132 at the first end 30 and the second end 32.

The tubular member 132 is thus C-shaped. The tubular member 132 here is substantially closed. The distance separating the ends 134, 136 is less than 80% of the maximum distance separating two opposite points located on the tubular member 132, on a line parallel to the line passing through the ends 134, 136.

The tubular member 132 thus forms a prosthetic valvular annulus able to be implanted surgically on the native mitral annulus.

As previously described, the shape of the native mitral annulus can be changed by actuating the adjusting mechanism 52 of the blocking member 26 in order to adjust the length of the central region 28 protruding outside the blocking member 26.

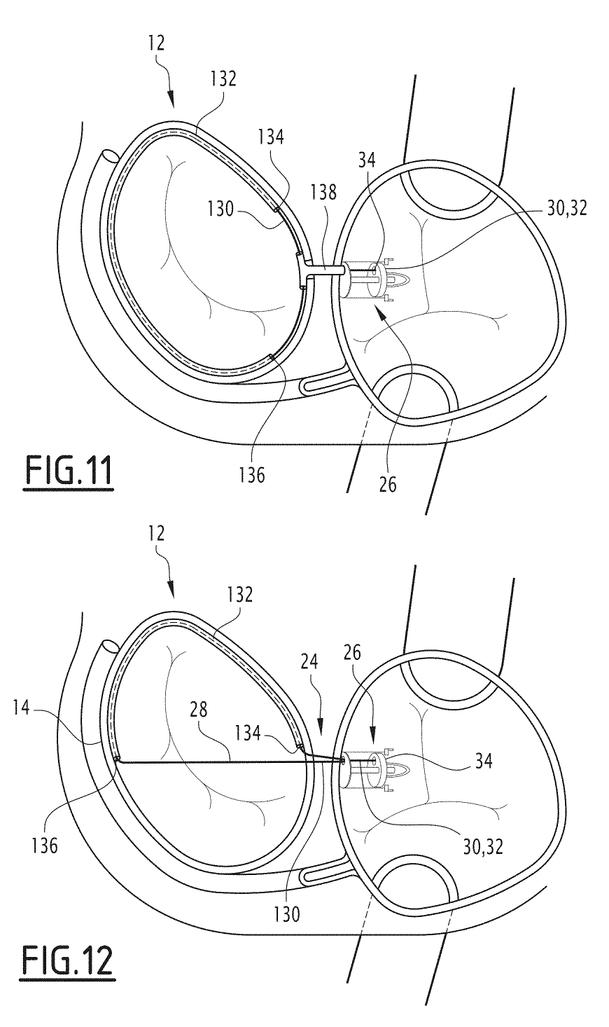

In the variant shown in FIG. 11, the shaping device 18 differs from that shown in FIG. 10 in that it includes a T-shaped guide 138 inserted between the ends 134, 136 of the tubular member 132 and the blocking member 26.

The T-shaped guide 138 is inserted through the interatrial septum. It guides the two opposite strands of the loop 130 toward the blocking member 26.

The shaping device 18 shown in FIG. 12 differs from that shown in FIG. 11 in that the tubular member 132 forms a prosthetic valvular semi-annulus.

The distance separating the ends 134, 136 is greater than 80% of the maximum distance separating two opposite points situated on the tubular member 132, on a line parallel to the line passing through the ends 134, 136, and is advantageously equal to the maximum distance separating two opposite points situated on the tubular member 132 on a line parallel to the line passing through the ends 134, 136.

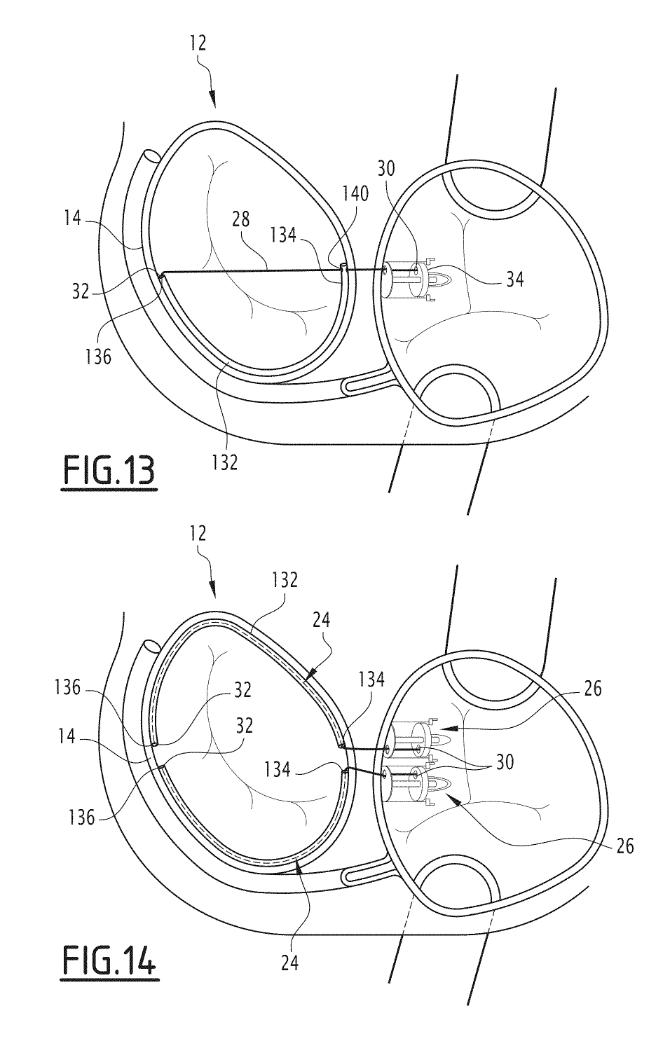

In the variant illustrated by FIG. 13, the elongate element 24 is not in a loop shape. The second end 32 of the filiform central region 28 is fixed to one end 136 of the tubular member 132. A guide passage 140 is arranged at the other end 134 of the tubular member 132. The central region 28 is engaged slidingly through the guide passage 140.

The adjustment of the length of the filiform central region 28 protruding outside the blocking member 26 causes the adjustment of the distance separating the ends 134, 136 of the tubular member 132 and therefore the shape of the prosthetic semi-annulus.

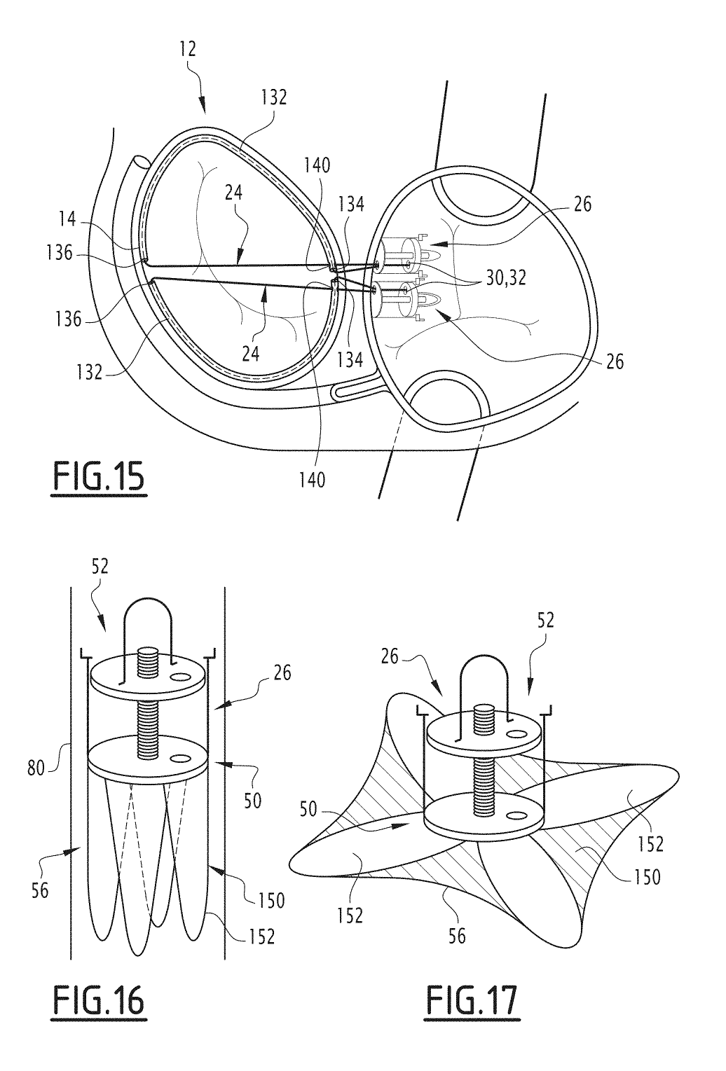

In the variants shown in FIGS. 14 and 15, two separate shaping devices 18 are mounted on the periphery of the native annulus.

In FIG. 14, each shaping device 18 includes an elongate element 24 inserted in the tubular member 132, with its second end 32 fixed to one end 136 of the tubular member 132.

In FIG. 15, the elongate element 24 is in the shape of a loop 130, a strand of which starting from the end 136 extends outside the tubular member 132, then passes through a guide passage 140 at the other end 134. A second strand of the loop 130 starting from the end 136 passes through the tubular member 132 and leaves the tubular member 132 at the other end-134.

In the variant illustrated in FIGS. 16 and 17, the patch 56 secured to the base 50 is deployable spontaneously between a retracted position, inserted in the catheter 80, and a position deployed outside the catheter 80.

The patch 56 here includes a deployable framework 150. The framework 150 is made up of a plurality of filiform loops 152 movable between an axial configuration, substantially parallel to the axis A-A′, and a transverse idle configuration, substantially perpendicular to the axis A-A′.

In the variant illustrated by FIG. 18, the fastening site 38 on the mobile stop 34 includes a thread 153 able to cooperate with a complementary thread 154 on the guide 90.

The guide 90 is thus able to be separated from the mobile stop 34 by unscrewing.

In the variant illustrated by FIG. 19, the adjusting mechanism 52 includes a rotary member 68 formed by a mobile shaft 160 mounted rotating about an axis B-B′ that is transverse relative to the axis A-A′ of the blocking member 26.

The mobile shaft 160 extends transversely in the axial passage 63 arranged in the guide tube 58. The axial passage 63 emerges through the passage orifice 62 on the one hand, and through an opposite orifice 164.

The fastening element 70 is arranged at an end of the mobile shaft 160 protruding outside the base 50.

The mobile stop 34 and the first end 30 of the filiform central region 28 are inserted through the axial passage to allow the winding around the shaft 160 by the rotational driving of the shaft 160 about the axis B-B′.

The rotation axis B-B′ is transverse to the local axis of the filiform central region 28 at the outlet of the blocking member 26.

In the variant illustrated in FIG. 20, the selective adjustment mechanism 52 includes a plurality of successive stops 170 distributed along the filiform central region 28, and a selective blocking system 172 arranged at the end of the base 50.

The blocking system 172 is able to move away during traction in a first direction (to the right in the figure) on the first end 30 to cause a stop 170 to pass beyond the blocking system 172. It is able to prevent the passage of each stop 170 when the filiform central region 28 moves in a second direction opposite the traction in the first direction.

The blocking system 172 here is formed by a plurality of resilient tabs 174 able to bend so as to converge toward the axis A-A′ when idle, and to move away from the axis A-A′ during the passage of the stop 170 in the first direction.

Unlike the device 18 shown in FIG. 1, the adjustment mechanism 52 of the shaping device 18 shown in FIG. 20 has no rotary member.

The adjustment of the length of the central region 28 protruding from the blocking member 26 toward the second end 32 is therefore done by traction on the elongate element 24 from the first end 30, to decrease the length of the central region 28 protruding outside the blocking member toward the second end 32.

In the variant shown in FIG. 21, the selective adjustment mechanism 52 includes a clamp 180 maneuverable between an open configuration allowing the passage of the successive stops 170 and a closed position allowing the blocking of the stops 170.

The operation is also similar to that of the device 18 of FIG. 20.

In the variant illustrated by FIG. 22, the shaping device 18 forms a prosthetic valvular chord.

Unlike the device 18 described in FIG. 1, the second end 32 of the filiform central region 28 is fixed on a clamp 200 mounted on an intracorporeal wall that here is a valve leaflet 16.

The intracorporeal clamp 200 is for example described in international application no. PCT/EP2015/077783 by the applicant. The second end 32 is fixed on the clamp 200, either before the implantation of the shaping device 18 in the patient, or during the placement of the clamp 200 on the intracorporeal wall. The second end 32 advantageously has no bearing plate 36.

The blocking member 26 is in turn applied on the inter-ventricular septum 201 on the side of the right ventricle.

In the variant illustrated by FIG. 23 and by FIG. 33, the blocking member 26 includes a deflector 210 protruding away from the adjusting mechanism 52 from a bearing surface 51 of the base 50, around the passage orifice 62.

The deflector 210 receives and guides an intermediate segment 212 of the central region 28. Thus, a first segment of the central region 28 comprising the first end 30 is received in the selective adjustment mechanism 52, as previously described. A second segment of the central region 28 comprising the second end 32, with a length adjustable by the selective adjusting mechanism 52, protrudes from the deflector 210.

In the example shown here, the deflector 210 is formed by a tube emerging at a first end opposite the passage orifice 62 and emerging transversely at a free end through a lateral opening 214.

In reference to FIG. 33, the length of the deflector 210 protruding from the bearing surface 51 of the base 50, taken along the axis A-A′, is greater than the length of the blocking member 26 protruding opposite the deflector 210, taken along the axis A-A′.

Furthermore, the deflector 210 here extends along an inclined direction relative to a normal N to the bearing surface 51, taken at the passage orifice 62. The incline angle I is for example between 1° and 45°.

During operation, the free end of the guide 90 is successively introduced into the deflector 210 by its free end 214, then into the blocking member 26. The elongate element 24 is partially introduced by its free end 30 through the free end 214 of the deflector 210, up to the passage orifice 62, then into the adjusting mechanism 52, as previously described.

The bearing surface 51 of the base 50 is then wedged against the septum 122 in the right atrium 123, while the deflector 212 protrudes in the left atrium 116 through the septum 122, in the vicinity of the mitral-aortic junction 216.

Thus, the segment of the elongate element 24 protruding outside the blocking member 26 from the deflector 212 extends substantially perpendicular to the separating line 218 between the leaflets 16, thus guaranteeing an excellent coaptation of the leaflets 16.

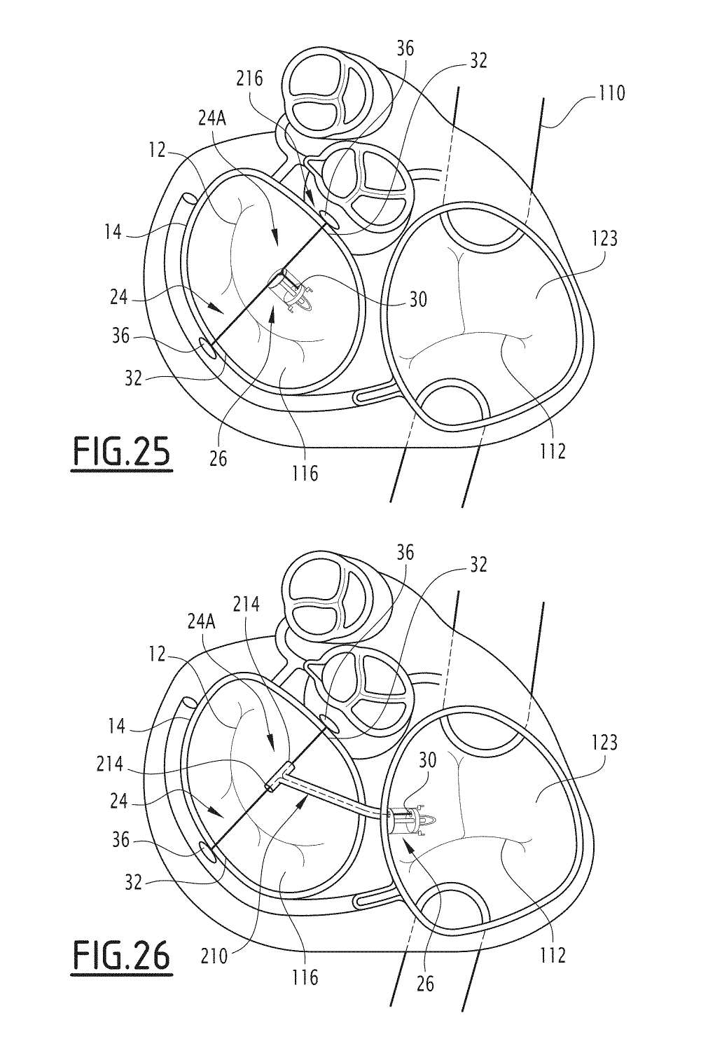

In the variant illustrated by FIGS. 24 and 25, the treatment kit 10 includes an additional elongate element 24A having a bearing plate 36 applied against the mitral-aortic junction 216, outside the left atrium 116.

Unlike the shaping device 18 previously described, the blocking member 26 is arranged directly in the left atrium 116 to connect the elongate element 24 and the additional elongate element 24A to one another by their first ends 30.

To that end, the first ends 30 provided with moving stops 34 are inserted into the blocking member 26 as previously described, by one or several passage orifices 62.

As illustrated by FIG. 24, the additional elongate element 24A is introduced using an additional guide 90A that is brought into the aorta, below the aortic valve to pass through the mitral-aortic junction 216. The grasping member 104 then simultaneously grasps the free ends 102 of each guide 90, 90A.

In the variant illustrated in FIG. 26, the blocking member 26 includes a deflector 210, as described in FIG. 23. However, unlike the deflector described in FIG. 23, the deflector 210 includes, at its free end, two lateral openings 214, 214A emerging opposite one another and respectively able to receive the elongate element 24 and the additional elongate element 24A.

In the variant illustrated by FIG. 27, the winder 64 includes a mobile member formed by a rotary shaft 230 that protrudes from the bearing surface 51 of the base 50, away from the fastening element 70.

The rotary shaft 230 is equipped at its free end with a fastening device 232 on a corporeal wall, here formed by the left atrium.

As illustrated in FIG. 29, the rotary shaft 230 is mounted rotating about the axis A-A′ relative to the base 50 and the fastening device 232, by driving using the fastening element 70.

The rotary shaft 230 is provided with at least one passage 72 for engagement of the central region 28 of an elongate element 24.

In the example shown in FIG. 29, the rotary shaft 230 includes a plurality of passages 72 emerging transversely relative to the axis A-A′.

The fastening device 232 includes at least one radially deployable anchor 234, provided here at its end with a hook 236. The anchor 234 is able to go spontaneously from a retracted configuration to a deployed configuration during the placement of the blocking member 26.

The fastening device 232 includes a skirt 238 for guiding the rotation of the shaft 230, which rotatably receives the free end of the shaft 230.

The blocking member 26 (in particular the rotary shaft 230 and the fastening device 232) advantageously delimits, over its entire length, an inner aperture for allowing the guide to pass, which allows its insertion into the patient.

During operation, the free end 124 of the guide 90, 90A associated with each elongate element 24, 24A is inserted through a respective passage 72 before the insertion of the blocking member 26 into the patient.

The blocking member 26 is then guided in the patient to place the base 50 bearing against the interatrial septum 122 in the right atrium 123.

The shaft 230 is inserted through the septum 122 in the left atrium 116. The fastening device 232 is then placed by deployment of the anchors 234, which fasten on the left atrium.

The rotation of the shaft 230 by actuation of the fastening element 70 then causes the winding or unwinding of a length of the central region 28 protruding from the blocking member 26 toward the second end 32.

In the example shown in FIG. 27, the device 18 comprises an elongate element 24 fastened on the wall of the left atrium 116 opposite the coronary sinus 114, on a side of the axis A-A′ and an additional elongate element 24A fixed on the mitral-aortic junction 216 on another side of the axis A-A′.

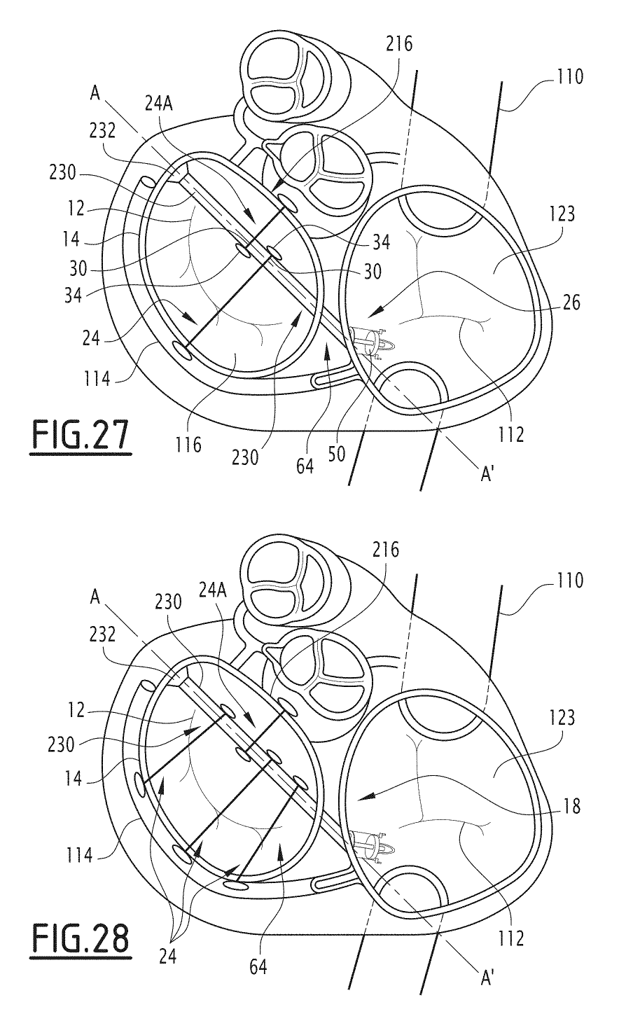

In the variant shown in FIG. 28, the device 18 comprises a plurality of elongate elements 24 fixed on the wall 14 of the left atrium opposite the coronary sinus 114, on one side of the axis A-A′, and a plurality of additional elongate elements 24A fixed on the mitral-aortic junction 216 on another side of the axis A-A′.

These devices 18 are very effective for guaranteeing a good coaptation of the leaflets 16 of the valve 12.

In the variant illustrated by FIG. 30, the shaft 230 is formed by two parallel resilient strips 250 defining a single engagement passage 72 between them.

The strips 250 are transversely deformable to open the passage 72, allowing the insertion of at least one engagement stop 34 of an elongate element 24.

To keep the strips 250 in their deformed position, a temporary wedge 252 is inserted between the strips 250 during the placement of the blocking member 26, as illustrated by FIG. 30.

Thus, once the shaft 230 is installed protruding in the left atrium 116, each end 30 of an elongate element 24 is inserted through the passage 72, the wedge 252 extending in place.

Then the wedge 252 is removed. The strips 250 return to their idle position, and lock each stop 34 in position between the strips 250, as illustrated by FIG. 31.

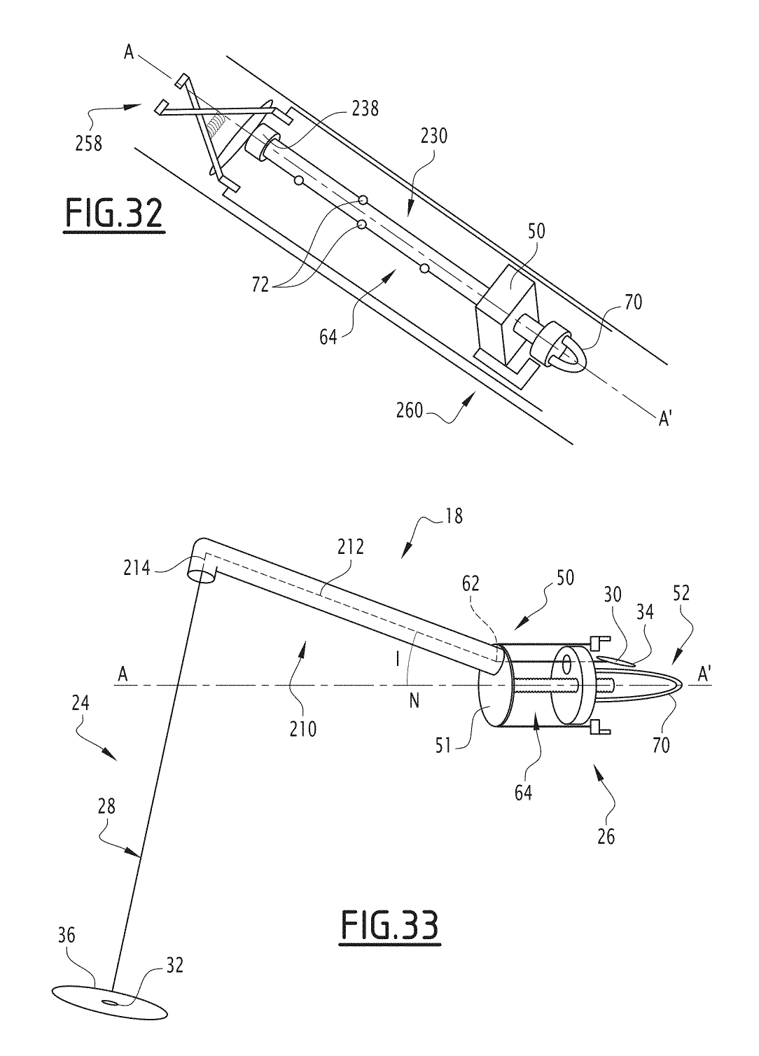

In the variant illustrated by FIG. 32, the fastening device 232 comprises a fastening clamp 258, kept open during the insertion of the blocking member 26 in the patient and which closes spontaneously to fasten the intracorporeal wall, once the blocking member 26 is released.

In the variants illustrated by FIGS. 29 to 32, the selective adjustment mechanism 52 advantageously includes a unidirectional rotational blocking system 260 for the winder 64 around the winding axis A-A′.

Such a blocking system 260 is for example a ratchet system. It is able to allow the rotation of the winder 64 in a first direction and to prevent the rotation of the winder 64 in a second direction.

This blocking system 260 can advantageously be installed on all of the blocking members 26 previously described.

The locking of the first end 30 of the elongate element 24 relative to the blocking member 26 and the adjustment by the adjusting mechanism 52 allow an adjustment of the length of the elongate element 24. For example, the adjustment is done by rotating the winder.

The blocking member 26 allows both the adjustment by the adjusting mechanism 52 and the locking making it possible to maintain this adjusting length.

Furthermore, the locking and the adjustment of the length are reversible.

Advantageously, the elongate element and the blocking member are radiopaque. The adjustment is made easier by the radiopacity of the elongate element 24 and the blocking member 26.

The blocking device 26 allowing both the setting and the adjustment is thus deployed by the endoluminal route. Furthermore, it remains accessible by the endoluminal route for later adjustments that may be repeated.

The adjustment mechanism 52 remains accessible and manipulatable using an actuating device 20 introduced by the endoluminal route. The selective adjustment mechanism 52 is an integral part of the blocking member 26.

1. An implantable device (18) for shaping an intracorporeal wall (14) including:
an elongate element (24) having a first end (30), a second end (32) and a central filiform region (28) between the first end (30) and the second end (32),
a blocking member (26), the elongate element (24) being mobile in relation to the blocking member (26) between an unlocked position away from the blocking member (26) and a locked position on the blocking member (26), in which the elongate element (24) is connected to the blocking member (26),
wherein the blocking member (26) includes a mechanism (52) for selective adjustment of the length of the central region (28) protruding towards the second end (32) from the blocking member (26).

2. The device (18) according to claim 1, wherein the blocking member (26) comprises a fixed base (50), delimiting a bearing surface (51) on an intracorporeal surface, the base (50) delimiting a passage orifice (62) for the elongate element (24), the central region (28) of the elongate element (24) protruding outside the blocking member (26) while passing through the passage orifice (62) in the locked position.

3. The device (18) according to claim 2, wherein the base (50) includes a deflector (210) protruding from the bearing surface (51) away from the selective adjusting mechanism (52), the deflector (210) guiding the central region (28) of the elongate element (24) from the passage orifice (62).

4. The device (18) according to claim 1, wherein the elongate element (24) is provided with at least one blocking stop (34), able to cooperate with the blocking member (26) in the locked position.

5. The device (18) according to claim 4, wherein the blocking stop (34) is mounted at the first end (30) or near the first end (30), and is maneuverable between an axial position for insertion in the blocking member (26) and a transverse position for connecting to the blocking member (26).

6. The device (18) according to claim 1, wherein the selective adjustment mechanism (52) includes a winder (64) able to wind part of the central region (28) of the elongate element (24) around a winding axis (A-A′; B-B′).

7. The device (18) according to claim 6, wherein the blocking member (26) comprises a fixed base (50), delimiting a bearing surface (51) on an intracorporeal surface, the winder (64) comprises a mobile member (68; 230), mounted rotating relative to the base (50) around the winding axis (A-A′; B-B′).

8. The device (18) according to claim 7, wherein the mobile member (68; 230) defines at least one through passage (72) through which the elongate element (24) passes in the locked position.

9. The device (18) according to claim 7, wherein the winder (64) includes an element (70) for fastening a maneuvering arm (82) rotatably to the mobile member (68).

10. The device (18) according to claim 6, wherein the selective adjustment mechanism (52) includes a unidirectional rotational blocking system (260) for the winder around the winding axis (A-A′; B-B′).

11. The device (18) according to claim 1, wherein the elongate element (24) is provided, at its second end (32), with a bearing plate (36) on an intracorporeal surface or a fastening clamp (200) on an intracorporeal surface.

12. The device (18) according to claim 1, wherein, in the locked position of the elongate element (24), the first end (30) and the second end (32) are mounted in the blocking member (26), the central region (28) forming a loop (130).

13. The device (18) according to claim 1, including a C-shaped tubular member (132), the elongate element (24) cooperating mechanically with the tubular member (132) to modify the shape of the tubular member (132) by selective adjustment of the length of the central region of the elongate element (24) using the selective adjustment mechanism (52) of the blocking member (26).

14. A treatment kit (10) comprising:
a shaping device (18) according to claim 1,
a device (20) for actuating the selective adjustment mechanism (52), maneuverable reversibly between an engaged configuration for actuating the selective adjustment mechanism (52) and a configuration detached from the selective adjusting mechanism (52).

15. The treatment kit (10) according to claim 14, wherein the actuating device (20) is intended to be introduced by the endoluminal route.

16. The kit (10) according to claim 14, wherein the actuating device (20) includes a catheter (80), able to be applied over the blocking member (26), and a mobile arm (82), able to grasp a fastening element (70) of the selective adjustment mechanism (52).

17. The kit (10) according to claim 14, comprising a device (22) for placement of the shaping device (18), the placement device (22) including a traction guide (90) for the first end (30) of the elongate element (24), able to be reversibly fixed on a fastening site (38) of the elongate element (24).

18. The device according to claim 2, wherein the elongate element is provided with at least one blocking stop, able to cooperate with the blocking member in the locked position.

19. The device according to claim 3, wherein the elongate element is provided with at least one blocking stop, able to cooperate with the blocking member in the locked position.

20. The device according to claim 2, wherein the selective adjustment mechanism includes a winder able to wind part of the central region of the elongate element around a winding axis.
